SEQUENCE LISTING (10 sequences)

SEQ ID NO: 1 - protein, 10 aa, Artificial Sequence
  B-701 H1-CDR
GFTFTSTGIS

SEQ ID NO: 2 - protein, 18 aa, Artificial Sequence
  B-701 H2-CDR
GRIYPTSGSTNYADSVKG

SEQ ID NO: 3 - protein, 20 aa, Artificial Sequence
  B-701 H3-CDR
ARTYGIYDLYVDYTEYVMDY

SEQ ID NO: 4 - protein, 11 aa, Artificial Sequence
  B-701 L1-CDR
RASQDVDTSLA

SEQ ID NO: 5 - protein, 7 aa, Artificial Sequence
  B-701 L2-CDR
SASFLYS

SEQ ID NO: 6 - protein, 9 aa, Artificial Sequence
  B-701 L3-CDR
QQSTGHPQT

SEQ ID NO: 7 - protein, 123 aa, Artificial Sequence
  B-701 heavy chain variable region
EVQLVESGGGLVQPGGSLRLSCAASGFTFTSTGISWVRQAPGKGLEWVGRIYPTSGSTNYADSVKGRFTISADTSKNTAYLQMNSLRAEDTAVYYCARTYGIYDLYVDYTEYVMDYWGQGTLV

SEQ ID NO: 8 - protein, 108 aa, Artificial Sequence
  B-701 light chain variable region
DIQMTQSPSSLSASVGDRVTITCRASQDVDTSLAWYKQKPGKAPKLLIYSASFLYSGVPSRFSGSGSGTDFTLTISSLQPEDFATYYCQQSTGHPQTFGQGTKVEIKR

SEQ ID NO: 9 - protein, 457 aa, Artificial Sequence
  B-701 heavy chain
EVQLVESGGGLVQPGGSLRLSCAASGFTFTSTGISWVRQAPGKGLEWVGRIYPTSGSTNYADSVKGRFTISADTSKNTAYLQMNSLRAEDTAVYYCARTYGIYDLYVDYTEYVMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, Artificial Sequence
  B-701 light chain
DIQMTQSPSSLSASVGDRVTITCRASQDVDTSLAWYKQKPGKAPKLLIYSASFLYSGVPSRFSGSGSGTDFTLTISSLQPEDFATYYCQQSTGHPQTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2016/0243228 A1
(43) Pub. Date: Aug. 25, 2016

(54) METHODS, COMPOSITIONS, AND KITS FOR TREATMENT OF CANCER

(71) Applicant: BIOCLIN THERAPEUTICS, INC., San Ramon, CA (US)

(72) Inventors: Jocelyn HOLASH, Alameda, CA (US); Stephen LAU, San Ramon, CA (US)

(21) Appl. No.: 15/048,030

(22) Filed: Feb. 19, 2016

Related U.S. Application Data
(60) Provisional application No. 62/150,235, filed on Apr. 20, 2015.
(60) Provisional application No. 62/118,350, filed on Feb. 19, 2015.

(51) Int. Cl. A61K 39/395; A61K 45/06

(52) U.S. Cl. CPC A61K 39/39558; A61K 45/06; A61K 2039/507

(57) ABSTRACT

Provided herein are the use of FGFR3 inhibitors and PD1 inhibitors to treat solid and hematologic cancers and compositions and kits comprising an FGFR3 inhibitor and a PD1 inhibitor.

### RELATED APPLICATIONS

The present application claims priority to U.S. Provisional Application No. 62/118,350, filed Feb. 19, 2015, and U.S. Provisional Application No. 62/150,235, filed Apr. 20, 2015, the disclosures of which are incorporated by reference herein in their entireties, including drawings.

### BACKGROUND

The present application is directed to methods, compositions, and kits that utilize a combination of FGFR3 inhibitor and PD1 inhibitor to treat cancer.

### SUMMARY

Provided herein in certain embodiments are methods of treating a solid or hematologic tumor in a subject in need thereof comprising administering a therapeutically effective amount of an FGFR3 inhibitor and a therapeutically effective amount of a PD1 inhibitor. In certain embodiments, the FGFR3 inhibitor binds FGFR3. In other embodiments, the FGFR3 inhibitor binds a ligand for FGFR3. In certain embodiments, the FGFR3 inhibitor is an antagonistic FGFR3 antibody, and in certain of these embodiments the antagonistic FGFR3 antibody comprises one or more of a CDR-H1 comprising SEQ ID NO:1, a CDR-H2 comprising SEQ ID NO:2, a CDR-H3 comprising SEQ ID NO:3, a heavy chain variable region comprising SEQ ID NO:7, a heavy chain comprising SEQ ID NO:9, a CDR-L1 comprising SEQ ID NO:4, a CDR-L2 comprising SEQ ID NO:5, a CDR-L3 comprising SEQ ID NO:6, a light chain variable region comprising SEQ ID NO:8, and a light chain comprising the amino acid sequence set forth in SEQ ID NO:10. In certain of these embodiments, the FGFR3 antagonistic antibody is B-701. In other embodiments, the antagonistic FGFR3 antibody is selected from the group consisting of PRO-001 and IMC-D11. In certain embodiments, the FGFR3 inhibitor is a small molecule pan-FGFR inhibitor, and in certain of these embodiments the pan-FGFR inhibitor is selected from the group consisting of infigratinib, AZD4547, LY2874455, Debio 1347, ARQ 087, and JNJ-42756493. In certain embodiments, the PD1 inhibitor binds PD1. In other embodiments, the PD1 inhibitor binds a ligand for PD1. In certain embodiments, the PD1 inhibitor is an antagonistic PD1 antibody, and in certain of these embodiments the antagonistic PD1 antibody is selected from the group consisting of nivolumab, pembrolizumab, CT-011, MEDI-0680, and RMP1-14. In other embodiments, the PD1 inhibitor is an antagonistic PD1 ligand antibody, and in certain of these embodiments the antagonistic PD1 ligand antibody is selected from the group consisting of MEDI-4736, RG7446, BMS-936559, MSB0010718C, and MPDL3280A.

Provided herein in certain embodiments are compositions comprising an FGFR3 inhibitor and a PD1 inhibitor. In certain of these embodiments, the compositions are pharmaceutical compositions, and in certain embodiments the compositions comprise one or more pharmaceutically acceptable carriers. In certain embodiments, the FGFR3 inhibitor binds FGFR3. In other embodiments, the FGFR3 inhibitor binds a ligand for FGFR3. In certain embodiments, the FGFR3 inhibitor is an antagonistic FGFR3 antibody, and in certain of these embodiments the antagonistic FGFR3 antibody comprises one or more of a CDR-H1 comprising SEQ ID NO:1, a CDR-H2 comprising SEQ ID NO:2, a CDR-H3 comprising SEQ ID NO:3, a heavy chain variable region comprising SEQ ID NO:7, a heavy chain comprising SEQ ID NO:9, a CDR-L1 comprising SEQ ID NO:4, a CDR-L2 comprising SEQ ID NO:5, a CDR-L3 comprising SEQ ID NO:6, a light chain variable region comprising SEQ ID NO:8, and a light chain comprising the amino acid sequence set forth in SEQ ID NO:10. In certain of these embodiments, the FGFR3 antagonistic antibody is B-701. In other embodiments, the antagonistic FGFR3 antibody is selected from the group consisting of PRO-001 and IMC-D11. In certain embodiments, the FGFR3 inhibitor is a small molecule pan-FGFR inhibitor, and in certain of these embodiments the pan-FGFR inhibitor is selected from the group consisting of infigratinib, AZD4547, LY2874455, Debio 1347, ARQ 087, and JNJ-42756493. In certain embodiments, the PD1 inhibitor binds PD1. In other embodiments, the PD1 inhibitor binds a ligand for PD1. In certain embodiments, the PD1 inhibitor is an antagonistic PD1 antibody, and in certain of these embodiments the antagonistic PD1 antibody is selected from the group consisting of nivolumab, pembrolizumab, CT-011, MEDI-0680, and RMP1-14. In other embodiments, the PD1 inhibitor is an antagonistic PD1 ligand antibody, and in certain of these embodiments the antagonistic PD1 ligand antibody is selected from the group consisting of MEDI-4736, RG7446, BMS-936559, MSB0010718C, and MPDL3280A.

Provided herein in certain embodiments are kits comprising an FGFR3 inhibitor and a PD1 inhibitor for use in treating cancer. In certain of these embodiments, the kits further comprise instructions for use.

Provided herein in certain embodiments is the use of an FGFR3 inhibitor and a PD1 inhibitor for use in formulating a medicament for the treatment of cancer. In certain of these embodiments, the FGFR3 inhibitor and PD1 inhibitor are formulated into a single medicament. In other embodiments, the FGFR3 inhibitor and PD1 inhibitor are formulated into separate medicaments which are administered in combination with one another.

### BRIEF DESCRIPTION OF DRAWINGS

FIG. 1: Changes in tumor volume in MC38 syngeneic tumor mice following administration of FGFR3 and/or PD1 inhibitor antibodies.

FIG. 2: A two week efficacy snapshot showing the changes in tumor volume in MC38 syngeneic tumor mice following administration of FGFR3 and/or PD1 inhibitor antibodies.

FIG. 3: Changes in tumor volume in mice implanted with Lewis Lung Carcinoma tumor cells following administration of FGFR3 and/or PD1 inhibitor antibodies.

FIG. 4: Changes in tumor volume in mice implanted with Madison 109 tumor cells following administration of FGFR3 and/or PD1 inhibitor antibodies.

### DETAILED DESCRIPTION

The following description of the invention is merely intended to illustrate various embodiments of the invention. As such, the specific modifications discussed are not to be construed as limitations on the scope of the invention. It will be apparent to one skilled in the art that various equivalents, changes, and modifications may be made without departing from the scope of the invention, and it is understood that such equivalent embodiments are to be included herein.

There are four single-pass transmembrane tyrosine kinase fibroblast growth factor receptors (FGFR1-4) in humans (Brooks 2012). FGFRs are overexpressed in many cancer types, often due to mutations that confer constitutive activation, making them an attractive target for therapeutic intervention. For example, the FGFR2b antibody FPA144 (FivePrime) is currently under development for the treatment of solid tumors, particularly gastric cancer. Other FGFR2 monoclonal antibodies in early development for cancer treatment include GP369 (Aveo) and HuGAL-FR21 (Galaxy) (Zhao 2010; Bai 2010). A humanized anti-FGFR4 has also been reported to inhibit tumor growth (Bumbaca 2011).

FGFR3 harbors both oncogenic and tumor suppressive properties. FGFR3 is frequently mutated in certain cancers, but in some normal tissues it can limit cell growth and promote cell differentiation (Lafitte 2013). The human FGFR3 antagonistic monoclonal antibody MFGR1877S (CAS No. 1312305-12-6), referred to herein as B-701 or BM2, was the first FGFR antibody to enter clinical development. B-701 is a lyophilized form of MGFR1877A. B-701 is currently in early development for the treatment of metastatic bladder cancer (urothelial cell carcinoma) and achondroplasia (dwarfism). B-701 was originally identified through phage display, then recombined with a human IgG1 backbone. B-701 binds with high affinity to both wild-type and mutant FGFR3, including the most prevalent mutations found in bladder cancer and achondroplasia (specifically, FGFR3-IIIb^{R248C}, FGFR3-IIIb^{K652E}, FGFR3-IIIb^{Y375C}, FGFR3-IIIb^{S249C}, and FGFR3-IIIb^{G372C}), while exhibiting no cross-reactivity with other FGFRs. B-701 was previously evaluated for safety in patients with t(4:14) translocated multiple myeloma (Clinical Trial NCT01122875). Other FGFR3 inhibitor antibodies currently in clinical or preclinical development include PRO-001 (Prochon) and IMC-D11 (ImClone). Additional FGFR3 antibodies for use in treating cancer and other diseases have been disclosed in, for example, U.S. Pat. No. 8,187,601 (Aveo) and U.S. Pat. No. 7,498,416 (Fibron).

Programmed cell death protein 1 (PD1) is an immune checkpoint receptor from the CD28 superfamily that limits T cell effector functions within tissues following activation by either of its two ligands, PDL1 or PDL2 (Pardoll 2012). PD1 downregulates the immune system by promoting apoptosis in antigen-specific T cells while reducing apoptosis in regulatory (i.e., suppressor) T cells. Certain tumor cells block anti-tumor immune responses in the tumor microenvironment by upregulating ligands for PD1. Blocking the PD1 pathway activates the immune system to attack tumors, and has been shown to induce sustained tumor regression in various tumor types. Accordingly, several PD1 antagonist antibodies are currently in various stages of clinical development. For example, the fully human IgG4 monoclonal PD1 antibody nivolumab (Opdivo®, Bristol-Myers Squibb and Ono Pharmaceutical; also known as ONO-4538, BMS-936558, MDX-1106) is approved for the treatment of unreselectable or metastatic melanoma in patients who no longer respond to other drugs. Nivolumab is also being evaluated for treatment of non-small cell lung cancer (NSCLC) in combination with various chemotherapy regimens. The humanized IgG4 PD1 antibody pembrolizumab (Keytruda®, Merck; also known as MK-3475) is approved for the treatment of melanoma. Other PD1 antibodies in development include CT-011 (Curetech) and MEDI-0680/AMP-514 (AstraZeneca).

A variety of PD1 ligand (PDL) antibodies are also in development for cancer treatment. For example, the monoclonal IgG1k PDL1 antibody MEDI-4736 (AstraZeneca) is currently in development for the treatment of NSCLC either alone or in combination with the monoclonal CTLA4 antibody tremelimumab (AstraZeneca) or MEDI-0680, the monoclonal IgG1k PDL1 antibody RG7446 (Roche) is in development for use in treating various cancers alone or in combination with Avastin® and Zelboraf®, the fully human monoclonal IgG4 antibody BMS-936559/MDX-1105 (BMS) is currently in development for the treatment of NSCLC and other cancer types, the fully human IgG1 PDL1 antibody MSB0010718C (Merck Serono) is in development for treating various cancer types, and the Fc-modified monoclonal IgG1 antibody MPDL3280A (Genentech) is currently in development for treatment of NSCLC.

As set forth in the Examples below, administration of an FGFR3 antagonist antibody in combination with a PD1 antagonist antibody resulted in slower tumor growth in MC38 syngeneic tumor model mice than administration of either antibody alone. These results are surprising because previous studies have shown that blockade of the FGFR3 pathway dampens the immune system rather than enhancing it (see, e.g., WO04/110487). Since the anti-cancer properties of PD1 inhibition are believed to derive from activation of the immune system to attack cancer cells, one of ordinary skill in the art would have expected FGFR3 inhibition to decrease the effectiveness of PD1 inhibition. Additionally, treatment with an FGFR3 antagonist antibody, B-701, resulted in a higher CD8+ cell to T regulatory cell ratio in MC38 tumor model mice, supporting the initial observation that B-701 can enhance efficacy of immune checkpoint inhibitors. The Examples below also describe administration of an FGFR3 antagonist antibody in combination with a PD1 antagonist antibody, which resulted in slower tumor growth in mice implanted with Madison 109 and Lewis Lung Carcinoma tumor cells than administration of either antibody alone. The present application provides practical applications of these findings in the form of compositions, methods, and kits for treating solid tumors using a combination of one or more FGFR3 inhibitors and one or more inhibitors of an immune checkpoint molecule.

Provided herein in certain embodiments are methods of treating a solid or hematologic cancer in a subject in need thereof comprising administering an FGFR3 inhibitor and a PD1 inhibitor. Also provided herein are methods of increasing the effectiveness of a PD1 inhibitor for treating cancer in a subject in need thereof comprising administering an FGFR3 inhibitor, as well as methods of increasing the effectiveness of an FGFR3 inhibitor for treating cancer in a subject in need thereof comprising administering a PD1 inhibitor. An increase in effectiveness of a PD1 or FGFR3 inhibitor may refer to an increase in the therapeutic effect of either inhibitor, a decrease in the required dosage, administration frequency, or administration interval of either inhibitor to obtain a particular level of therapeutic effect, or some combination thereof.

The term “solid cancer” as used herein refers to a cancer that forms a discrete tumor mass. Examples of solid cancers within the scope of the present methods include cancers of the colon, rectum, kidney, bladder, prostate, brain, breast, liver, lung, skin (e.g., melanoma), and head and neck.

The term “hematologic cancer” as used herein refers to cancers that occur in cells of the immune system or in blood-forming tissues including bone marrow and which generally do not form solid tumors. Examples of hematologic cancers within the scope of the present methods include leukemia (e.g., acute myeloid leukemia, acute lymphoblastic leukemia, chronic myelogenous leukemia, and chronic lymphocytic leukemia), Hodgkin and non-Hodgkin lymphoma, myeloma, and myelodysplastic syndrome.

The terms “treat,” “treating,” and “treatment” as used herein with regard to solid cancers may refer to partial or total inhibition of tumor growth, reduction of tumor size, complete or partial tumor eradication, reduction or prevention of malignant growth, partial or total eradication of cancer cells, or some combination thereof. The terms “treat,” “treating,” and “treatment” as used herein with regard to hematological cancers may refer to complete or partial regression or remission, prevention, slowing, or reduction of cancer remission, partial or total eradication of cancer cells, or some combination thereof. The phrases “patient” and “subject” are used interchangeably herein.

A “subject in need thereof” as used herein refers to a mammalian subject, preferably a human, who has been diagnosed with solid or hematologic cancer, is suspected of having solid or hematologic cancer, and/or exhibits one or more symptoms associated with solid or hematologic cancer. In certain embodiments, the subject may have previously received one or more therapeutic interventions for the treatment of cancer, e.g., chemotherapy.

An “FGFR3 inhibitor” as used herein refers to any molecule that inhibits the activity of FGFR3 either partially or completely. An FGFR3 inhibitor may inhibit FGFR3 specifically, or it may inhibit the activity of other proteins in addition to FGFR3. For example, an FGFR3 inhibitor may also inhibit the activity of other FGFRs.

In certain embodiments of the methods, compositions, and kits provided herein, the FGFR3 inhibitor inhibits FGFR3 activity by binding to FGFR3. Examples of such FGFR3 inhibitors include, for example, antagonistic FGFR3 antibodies or fusion proteins thereof, inactive forms of the FGFR3 ligand (e.g., truncated or otherwise mutated forms of the FGFR3 ligand) or fusion proteins thereof, small molecules, siRNAs, and aptamers. In certain of these embodiments, the FGFR3 inhibitor specifically binds FGFR3, meaning that the inhibitor exhibits little or no binding to other FGFRs. In other embodiments, the FGFR3 inhibitor binds one or more FGFRs in addition to FGFR3.

In certain preferred embodiments of the methods, compositions, and kits provided herein, the FGFR3 inhibitor is an FGFR3 antagonist antibody, and in certain of these embodiments the FGFR3 antagonist antibody specifically binds FGFR3. The term “antibody” as used herein refers to an immunoglobulin molecule or an immunologically active portion thereof that binds to a specific antigen, for example FGFR3 or PD1. In those embodiments wherein an for use in the present methods, compositions, and kits is a full-length immunoglobulin molecule, the antibody comprises two heavy chains and two light chains, with each heavy and light chain containing three complementary determining regions (CDRs). In those embodiments wherein an antibody is an immunologically active portion of an immunoglobulin molecule, the antibody may be, for example, a Fab, Fab′, Fv, Fab′ F(ab′)₂, disulfide-linked Fv, scFv, single domain antibody (dAb), or a diabody. Antibodies for use in the present methods, compositions, and kits may include natural antibodies, synthetic antibodies, monoclonal antibodies, polyclonal antibodies, chimeric antibodies, humanized antibodies, multispecific antibodies, bispecific antibodies, dual-specific antibodies, anti-idiotypic antibodies, or fragments thereof that retain the ability to bind a specific antigen, for example FGFR3 or PD1. Exemplary antibodies include IgA, IgD, IgG1, IgG2, IgG3, IgM and the like. In certain preferred embodiments of the methods, compositions, and kits provided herein, an FGFR3 antibody is an IgG2 antibody.

In certain embodiments, an FGFR3 antagonist antibody for use in the present methods, compositions, and kits comprises a heavy chain variable region comprising one or more complementary determining regions (CDRs) having the sequences set forth in SEQ ID NOs:1-3. In certain of these embodiments, the FGFR3 antagonist antibody comprises all three of these CDR sequences, and in certain of these embodiments the FGFR3 antagonist antibody comprises a heavy chain variable region comprising the amino acid sequence set forth in SEQ ID NO:4. In certain embodiments, the FGFR3 antagonist antibody comprises a light chain variable region comprising one or more CDRs having the sequences set forth in SEQ ID NOs:5-7. In certain of these embodiments, the FGFR3 antagonist antibody comprises all three of these CDR sequences, and in certain of these embodiments the FGFR3 antagonist antibody comprises a light chain variable region comprising the amino acid sequence set forth in SEQ ID NO:8. In certain embodiments, the FGFR3 antagonist antibody comprises all six CDR sequences set forth in SEQ ID NOs:1-3 and 5-7, and in certain of these embodiments the FGFR3 antagonist antibody comprises the heavy chain variable region of SEQ ID NO:4 and the light chain variable region of SEQ ID NO:8. In certain embodiments, the antibody is B-701 comprising the heavy chain of SEQ ID NO:9 and the light chain of SEQ ID NO:10. In addition to the variable region set forth in SEQ ID NO:7, the heavy chain SEQ ID NO:9 comprises human IgG1. Similarly, the light chain of SEQ ID NO:10 comprises the variable region set forth in SEQ ID NO:8 and human Ig kappa chain C (UniProt P01834).

SEQ ID NO: 1 (H1-CDR):
GFTFTSTGIS.
SEQ ID NO: 2 (H2-CDR):
GRIYPTSGSTNYADSVKG.
SEQ ID NO: 3 (H3-CDR):
ARTYGIYDLYVDYTEYVMDY.
SEQ ID NO: 4 (L1-CDR):
RASQDVDTSLA.
SEQ ID NO: 5 (L2-CDR):
SASFLYS.
SEQ ID NO: 6 (L3-CDR):
QQSTGHPQT.
SEQ ID NO: 7:
EVQLVESGGGLVQPGGSLRLSCAASGFTFTSTGISWVRQAPGKGL
EWVGRIYPTSGSTNYADSVKGRFTISADTSKNTAYLQMNSLRAED
TAVYYCARTYGIYDLYVDYTEYVMDYWGQGTLV.
SEQ ID NO: 8:
DIQMTQSPSSLSASVGDRVTITCRASQDVDTSLAWYKQKPGKAPK
LLIYSASFLYSGVPSRFSGSGSGTDFTLTISSLQPEDFATYYCQQ
STGHPQTFGQGTKVEIKR.
SEQ ID NO: 9:
EVQLVESGGGLVQPGGSLRLSCAASGFTFTSTGISWVRQAPGKGL
EWVGRIYPTSGSTNYADSVKGRFTISADTSKNTAYLQMNSLRAED
TAVYYCARTYGIYDLYVDYTEYVMDYWGQGTLVTVSSASTKGPSV
FPLAPSSKSTSGGTAALGCLVKDYFPEPVTVSWNSGALTSGVHTF
PAVLQSSGLYSLSSVVTVPSSSLGTQTYICNVNHKPSNTKVDKKV
EPKSCDKTHTCPPCPAPELLGGPSVFLFPPKPKDTLMISRTPEVT
CVVVDVSHEDPEVKFNWYVDGVEVHNAKTKPREEQYNSTYRVVSV
LTVLHQDWLNGKEYKCKVSNKALPAPIEKTISKAKGQPREPQVYT
LPPSREEMTKNQVSLTCLVKGFYPSDIAVEWESNGQPENNYKTTP
PVLDSDGSFFLYSKLTVDKSRWQQGNVFSCSVMHEALHNHYTQKS
LSLSPGK.
SEQ ID NO: 10:
DIQMTQSPSSLSASVGDRVTITCRASQDVDTSLAWYKQKPGKAPK
LLIYSASFLYSGVPSRFSGSGSGTDFTLTISSLQPEDFATYYCQQ
STGHPQTFGQGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC.

In other embodiments, an FGFR3 antagonist antibody for use in the present methods, compositions, and kits may be PRO-001, IMC-D11, or an FGFR3 antagonistic antibody as disclosed in U.S. Pat. No. 8,187,601 (Aveo) or U.S. Pat. No. 7,498,416 (Fibron).

In certain embodiments of the methods, compositions, and kits provided herein, the FGFR3 inhibitor inhibits FGFR3 activity by binding to a ligand for FGFR3. Examples of such FGFR3 inhibitors include, for example, antibodies that specifically bind an FGFR3 ligand or fusion proteins thereof, soluble forms of FGFR3 comprising all or part of the FGFR3 extracellular domain or fusion proteins thereof, truncated forms of FGFR3 lacking all or part of the intracellular domains required for downstream signaling or fusion proteins thereof, small molecules, siRNAs, and aptamers.

In certain embodiments of the methods, compositions, and kits provided herein, the FGFR3 inhibitor is a pan-FGFR inhibitor, meaning that it binds to and inhibits the activity of one or more FGFRs in addition to FGFR3. In certain of these embodiments, the FGFR3 inhibitor may be a small molecule pan-FGFR inhibitor selected from the group consisting of infigratinib (BGJ398, Novartis), AZD4547 (AstraZeneca), LY2874455 (Eli Lilly), Debio 1347 (Debiopharm), ARQ 087 (ArQule), JNJ-42756493 (Janssen), and PRN1371 (Principia).

In certain embodiments of the methods, compositions, and kits provided herein, the FGFR3 inhibitor inhibits FGFR3 activity by blocking downstream tyrosine kinase activity. For example, a non-selective tyrosine kinase inhibitor such as dovitinib, lucitinib, ponatinib, nintedanib, ponatinib, or ENMD-2076 may be utilized as an FGFR3 inhibitor.

A “PD1 inhibitor” as used herein refers to any molecule that inhibits the activity of PD1 either partially or completely. A PD1 inhibitor may inhibit PD1 specifically, or it may inhibit the activity of other proteins in addition to PD1. For example, a PD1 inhibitor may also inhibit the activity of other immune checkpoint molecules.

In certain embodiments of the methods, compositions, and kits provided herein, the PD1 inhibitor inhibits PD1 activity by binding to PD1. Examples of such PD1 inhibitors include, for example, antagonistic PD1 antibodies or fusion proteins thereof, inactive forms of a PD1 ligand (e.g., truncated or otherwise mutated forms of PDL1 or PDL2) or fusion proteins thereof (e.g., AMP-224 (GlaxoSmithKline, Amplimmune), small molecules, siRNAs, and aptamers.

In certain embodiments of the methods, compositions, and kits provided herein, the PD1 inhibitor is a PD1 antibody, and in certain of these embodiments the PD1 antagonist antibody specifically binds PD1. In certain embodiments, the PD1 antagonistic antibody is selected from the group consisting of nivolumab, pembrolizumab, CT-011, MEDI-0680, and RMP1-14.

In certain embodiments of the methods, compositions, and kits provided herein, the PD1 inhibitor inhibits PD1 activity by binding to one or more ligands for PD1, i.e., PDL1 or PDL2. Examples of such PD1 inhibitors include, for example, PD1 ligand antibodies or fusion proteins thereof, soluble forms of PD1 comprising all or part of the PD1 extracellular domain or fusion proteins thereof, truncated forms of PD1 lacking all or part of the intracellular domains required for downstream signaling or fusion proteins thereof, small molecules, siRNAs, and aptamers.

In certain embodiments of the methods, compositions, and kits provided herein, the PD1 inhibitor is a PD1 ligand antibody, and in certain of these embodiments the PD1 ligand antibody specifically binds the PD1 ligand. In certain embodiments, the PD1 ligand antibody is selected from the group consisting of MEDI-4736, RG7446, BMS-936559, MSB0010718C, and MPDL3280A.

In certain embodiments of the methods provided herein, the FGFR3 inhibitor and PD1 inhibitor are administered together as part of the same composition. In other embodiments, the FGFR3 inhibitor and PD1 inhibitor are administered separately, i.e., as separate compositions. In these embodiments, the inhibitors may be administered simultaneously or sequentially, and may be administered via the same or different routes. In those embodiments where the inhibitors are administered sequentially, they may be administered at the same or different intervals. For example, one inhibitor may be administered more frequently than the other, or may be administered over a longer time course. In certain of these embodiments, one inhibitor may be administered one or more times prior to the first administration of the second inhibitor. When administration of the second inhibitor is initiated, administration of the first inhibitor may either cease or continue for all or part of the course of administration of the second inhibitor. In certain embodiments wherein the FGFR3 inhibitor is an FGFR3 antagonist antibody, the antibody may be administered two or more times per day, daily, two or more times per week, weekly, bi-weekly (i.e., every other week), every third week, or monthly. In certain embodiments, the antibody is administered weekly, bi-weekly, or every third week. In certain embodiments wherein the PD1 inhibitor is a PD1 antagonist antibody, the antibody may be administered two or more times per day, daily, two or more times per week, weekly, bi-weekly, every third week, or monthly. In certain embodiments, the PD1 inhibitor is administered bi-weekly. In certain embodiments, the FGFR3 inhibitor and/or the PD1 inhibitor may be administered for a specific time course determined in advance. For example, the FGFR3 and/or PD1 inhibitors may be administered for a time course of 1 day, 2 days, 1 week, 2 weeks, 4 weeks, or 8 weeks. In other embodiments, the FGFR3 and/or PD1 inhibitors may be administered indefinitely, or until a specific therapeutic benchmark is reached. For example, the FGFR3 and/or PD1 inhibitors may be administered until tumor growth is arrested or reversed, until one or more tumors are eliminated, or until the number of cancer cells are reduced to a specific level.

A “therapeutically effective amount” of a composition as used herein is an amount of a composition that produces a desired therapeutic effect in a subject, such as treating cancer. In certain embodiments, the therapeutically effective amount is an amount of the composition that yields maximum therapeutic effect. In other embodiments, the therapeutically effective amount yields a therapeutic effect that is less than the maximum therapeutic effect. For example, a therapeutically effective amount may be an amount that produces a therapeutic effect while avoiding one or more side effects associated with a dosage that yields maximum therapeutic effect. A therapeutically effective amount for a particular composition will vary based on a variety of factors, including but not limited to the characteristics of the therapeutic composition (e.g., activity, pharmacokinetics, pharmacodynamics, and bioavailability), the physiological condition of the subject (e.g., age, body weight, sex, disease type and stage, medical history, general physical condition, responsiveness to a given dosage, and other present medications), the nature of any pharmaceutically acceptable carriers in the composition, and the route of administration. One skilled in the clinical and pharmacological arts will be able to determine a therapeutically effective amount through routine experimentation, namely by monitoring a subject's response to administration of a composition and adjusting the dosage accordingly. For additional guidance, see, e.g., Remington: The Science and Practice of Pharmacy, 22^{nd} Edition, Pharmaceutical Press, London, 2012, and Goodman & Gilman's The Pharmacological Basis of Therapeutics, 12^{th} Edition, McGraw-Hill, New York, N.Y., 2011, the entire disclosures of which are incorporated by reference herein.

In certain embodiments of the methods provided herein, a therapeutically effective amount of an FGFR3 inhibitor or a PD1 inhibitor may be a dosage at which the molecule is capable of generating a therapeutic response (e.g., reducing or eliminating tumor growth) as a monotherapy, i.e., when administered alone. In certain of these embodiments, the therapeutically effective amount may be a dosage that has previously been determined to be optimal or near optimal for cancer treatment. For example, where the FGFR3 inhibitor is B-701, the antibody may be administered at a dosage of about 10 to 50 mg/kg every two to four weeks, and in certain of these embodiments the antibody may be administered at a dosage of about 20 to 40 mg/kg every two to four weeks, or about 30 mg/kg every three weeks. In other embodiments, a therapeutically effective amount of an FGFR3 inhibitor or a PD1 inhibitor may be lower than the dosage at which the molecule would normally be administered for use as a monotherapy, i.e., a suboptimal dose. In certain of these embodiments, administration of the suboptimal dosage of FGFR3 or PD1 inhibitor may result in decreased side effects versus the standard dosage when administered alone. For example, administration of suboptimal dosage of FGFR3 or PD1 inhibitors may result in decreased occurrence or severity of pruritus, colitis, or pneumonia versus administration of the optimal dosage of either inhibitor alone. In certain embodiments, one of an FGFR3 inhibitor and a PD1 inhibitor may be administered at a dosage that has been determined to be optimal for cancer treatment when administered alone, while the other is administered at a dosage that is suboptimal for treatment when administered alone. In certain embodiments, the dosage of the FGFR3 inhibitor or PD1 inhibitor may change over the course of the treatment regimen. For example, one or both of the FGFR3 inhibitor and PD1 inhibitor may be administered at higher dosage at the start of treatment (e.g., a loading phase), followed by a lower dosage later in treatment.

An FGFR3 inhibitor, PD1 inhibitor, or composition comprising both an FGFR3 inhibitor and a PD1 inhibitor may be delivered to a subject by any administration pathway known in the art, including but not limited to parenteral, oral, aerosol, enteral, nasal, ophthalmic, parenteral, or transdermal (e.g., topical cream or ointment, patch). “Parenteral” refers to a route of administration that is generally associated with injection, including intravenous, intraperitoneal, subcutaneous, infraorbital, infusion, intraarterial, intracapsular, intracardiac, intradermal, intramuscular, intrapulmonary, intraspinal, intrasternal, intrathecal, intrauterine, subarachnoid, subcapsular, transmucosal, or transtracheal. In certain embodiments wherein the FGFR3 inhibitor is an FGFR3 antagonist antibody, including for example B-701, the FGFR3 inhibitor is administered intravenously. In certain embodiments wherein the PD1 inhibitor is a PD1 antagonist antibody, the PD1 inhibitor is administered intraperitoneally.

In certain embodiments, FGFR3 inhibitors, PD1 inhibitors, or compositions comprising both FGFR3 and PD1 inhibitors may be formed into oral dosage units, such as for example tablets, pills, or capsules. In certain embodiments, FGFR3 inhibitor, PD1 inhibitor, or FGFR3 and PD1 inhibitor compositions may be administered via a time release delivery vehicle, such as, for example, a time release capsule. A “time release vehicle” as used herein refers to any delivery vehicle that releases active agent over a period of time rather than immediately upon administration. In other embodiments, FGFR3 inhibitor, PD1 inhibitor, or FGFR3 and PD1 inhibitor compositions may be administered via an immediate release delivery vehicle.

In certain embodiments of the methods provided herein, subjects receiving FGFR3 inhibitor and PD1 inhibitor may receive additional therapies, including for example chemotherapy or immunotherapy, before, during, or after treatment with FGFR3 and PD1 inhibitors. In those embodiments where the subject receives additional therapies during treatment with FGFR3 and PD1 inhibitors, the additional therapies may be administered simultaneously or sequentially with the FGFR3 inhibitor and/or PD1 inhibitor.

Provided herein in certain embodiments are compositions comprising a therapeutically effective amount of an FGFR3 inhibitor and a therapeutically effective amount of a PD1 inhibitor. In certain embodiments, these compositions further comprise one or more pharmaceutically acceptable carriers, or are formulated for administration with one or more pharmaceutically acceptable carriers. Also provided herein are kits comprising an FGFR3 inhibitor and a PD1 inhibitor for use in carrying out the methods disclosed herein, e.g., for treating cancer.

In certain embodiments of the compositions and kits provided herein, an FGFR3 inhibitor or PD1 inhibitor may be present in the composition or kit at a dosage at which it is capable of generating a therapeutic response (e.g., reducing or eliminating tumor growth) when administered alone. In certain of these embodiments, the FGFR3 or PD1 inhibitor may be present at a dosage that has previously been determined to be optimal or near optimal for cancer treatment. For example, where the FGFR3 inhibitor is B-701, the composition or kit may be formulated to deliver a dosage of about 10 to 50 mg/kg of B-701 to the subject, and in certain of these embodiments the composition or kit may be formulated to deliver a dosage of about 20 to 40 mg/kg or about 30 mg/kg of B-701 to the subject. In other embodiments, the FGFR3 or PD1 inhibitor may be present at a dosage that is lower than that at which it would normally be present in a composition or kit for cancer treatment (i.e., a suboptimal dose).

A “pharmaceutically acceptable carrier” as used herein refers to a pharmaceutically acceptable material, composition, or vehicle that is involved in carrying or transporting a compound or molecule of interest from one tissue, organ, or portion of the body to another tissue, organ, or portion of the body. A pharmaceutically acceptable carrier may comprise a variety of components, including but not limited to a liquid or solid filler, diluent, excipient, solvent, buffer, encapsulating material, surfactant, stabilizing agent, binder, or pigment, or some combination thereof. Each component of the carrier must be “pharmaceutically acceptable” in that it must be compatible with the other ingredients of the composition and must be suitable for contact with any tissue, organ, or portion of the body that it may encounter, meaning that it must not carry a risk of toxicity, irritation, allergic response, immunogenicity, or any other complication that excessively outweighs its therapeutic benefits.

Examples of pharmaceutically acceptable carriers that may be used in conjunction with the compositions provided herein include, but are not limited to, (1) sugars, such as lactose, glucose, sucrose, or mannitol; (2) starches, such as corn starch and potato starch; (3) cellulose and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols such as propylene glycol; (11) polyols such as glycerin, sorbitol, mannitol and polyethylene glycol; (12) esters, such as ethyl oleate and ethyl laurate; (13) disintegrating agents such as agar or calcium carbonate; (14) buffering or pH adjusting agents such as magnesium hydroxide, aluminum hydroxide, sodium chloride, sodium lactate, calcium chloride, and phosphate buffer solutions; (15) alginic acid; (16) pyrogen-free water; (17) isotonic saline; (18) Ringer's solution; (19) alcohols such as ethyl alcohol and propane alcohol; (20) paraffin; (21) lubricants, such as talc, calcium stearate, magnesium stearate, solid polyethylene glycol, or sodium lauryl sulfate; (22) coloring agents or pigments; (23) glidants such as colloidal silicon dioxide, talc, and starch or tri-basic calcium phosphate; (24) other non-toxic compatible substances employed in pharmaceutical compositions such as acetone; and (25) combinations thereof.

Compositions comprising an FGFR3 inhibitor, a PD1 inhibitor, or a combination of an FGFR3 inhibitor and a PD1 inhibitor may be formulated into a suitable dosage form, including for example solutions or suspensions in an aqueous or non-aqueous liquid, oil-in-water or water-in-oil liquid emulsions, capsules, cachets, pills, tablets, lozenges, powders, granules, elixirs or syrups, or pastilles. In certain embodiments, the compositions may be formulated as time release delivery vehicles, such as, for example, a time release capsule. A “time release vehicle” as used herein refers to any delivery vehicle that releases an active agent over a period of time rather than immediately upon administration. In other embodiments, the compositions may be formulated as immediate release delivery vehicles.

Provided herein in certain embodiments are kits for carrying out the methods disclosed herein. In certain embodiments, the kits provided herein comprise an FGFR3 inhibitor and a PD1 inhibitor. In certain embodiments, the FGFR3 inhibitor and PD1 inhibitor may be present in the kit in a single composition. In other embodiments, the FGFR3 inhibitor and PD1 inhibitor may be present in separate compositions. The kits may comprise additional therapeutic or non-therapeutic compositions. In certain embodiments, the kits comprise instructions in a tangible medium.

Provided herein in certain embodiments are an FGFR3 inhibitor and a PD1 inhibitor for use in the treatment of cancer. Also provided are an FGFR3 inhibitor for use in the treatment of cancer in combination with a PD1 inhibitor, and a PD1 inhibitor for use in the treatment of cancer in combination with an FGFR3 inhibitor.

Provided herein in certain embodiments is the use of an FGFR3 inhibitor and a PD1 inhibitor in the manufacture of a medicament for treating cancer. Also provided are the use of an FGFR3 inhibitor in the manufacture of a medicament for treating cancer in combination with a PD1 inhibitor, and the use of a PD1 inhibitor in the manufacture of a medicament for treating cancer in combination with an FGFR3 inhibitor.

The term “about” as used herein means within 10% of a stated value or range of values.

One of ordinary skill in the art will recognize that the various embodiments described herein can be combined. For example, steps from the various methods of treatment disclosed herein may be combined in order to achieve a satisfactory or improved level of treatment.

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention. It will be understood that many variations can be made in the procedures herein described while still remaining within the bounds of the present invention. It is the intention of the inventors that such variations are included within the scope of the invention.

### EXAMPLES

### Example 1

### Effect of FGFR3 and Immune Checkpoint Antagonists on Solid Tumor Development

FGFR3 expression was verified in the FGFR3-positive MC38 mouse colorectal tumor cell line using a commercially available ELISA kit for FGFR3. Subsequently the cell line was expanded and 1×10⁶ MC38 tumor cells were implanted subcutaneously into the flanks of female C57BL/6 mice that were 8 to 12 weeks of age. When tumors reached an average size of 80-120 mm³, animals were pair matched and treatment was initiated as described in Table 1.

TABLE 1
MC38 treatment regimen

| | | Regimen 1 | | | | Regimen 2 | | | | Regimen 3 | | | |
|---|---|---|---|---|---|---|---|---|---|---|---|---|---|
| Gr. | N | Agent | mg/kg | Route | Schedule | Agent | mg/kg | Route | Schedule | Agent | mg/kg | Route | Schedule |
| 1^{#} | 9 | PBS | — | iv | biwk × 3 | — | — | — | — | — | — | — | — |
| 2 | 9 | anti-PD1 | 5 | ip | biwk × 2 | — | — | — | — | — | — | — | — |
| | | RMP1-14 | | | | | | | | | | | |
| 3 | 9 | BM2 | 30 | iv | biwk × 3 | — | — | — | — | — | — | — | — |
| 4 | 9 | BM2 | 50 | iv | qwk × 3 | — | — | — | — | — | — | — | — |
| 5 | 9 | anti-PD1 | 5 | ip | biwk × 2 | BM2 | 30 | iv | biwk × 3 | — | — | — | — |
| | | RMP1-14 | | | | | | | | | | | |
| 6 | 9 | anti-PD1 | 5 | ip | biwk × 2 | BM2 | 50 | iv | qwk × 3 | — | — | — | — |
| | | RMP1-14 | | | | | | | | | | | |
| 7 | 9 | BM2 | 50 | iv | qwk × 3 | | — | — | — | — | — | — | — |
| | | | | | (start on | | | | | | | | |
| | | | | | day 8) | | | | | | | | |
| 8 | 9 | anti-PD1 | 5 | ip | biwk × 2 | BM2 | 50 | iv | qwk × 3 | — | — | — | — |
| | | RMP1-14 | | | | | | | (start on | | | | |
| | | | | | | | | | day 8) | | | | |
| 9 | 9 | anti- | 5 | ip | day 1 | anti- | 2.5 | ip | days 4, 7 | — | — | — | — |
| | | CTLA4 | | | | CTLA4 | | | | | | | |
| | | 9H10 | | | | 9H10 | | | | | | | |
| 10 | 9 | anti- | 5 | ip | day 1 | anti- | 2.5 | ip | days 4, 7 | anti- | 5 | ip | biwk × 2 |
| | | CTLA4 | | | | CTLA4 | | | | PD1 | | | |
| | | 9H10 | | | | 9H10 | | | | RMP1- | | | |
| | | | | | | | | | | 14 | | | |

As set forth in Table 1, Group 1 control mice received PBS via twice weekly (“biwk”) intravenous administration. Group 2 mice received RMP1-14, a rat anti-PD1 monoclonal antibody, twice weekly via interperitoneal administration at 5 mg/kg, while Group 3, 4, and 7 received BM2, a human anti-FGFR3 monoclonal antibody, either weekly (“qwk”) or twice weekly via intravenous administration at 30 mg/kg or 50 mg/kg. Groups 5, 6, and 8 received 5 mg/kg RMP1-14 in combination with BM2 at various dosages and frequencies. Group 9 mice received 9H10, a mouse anti-cytotoxic T-lymphocyte-associated antigen (CTLA4) monoclonal antibody via intraperitoneal administration on days 1, 4, and 7, and Group 10 received 9H10 and RMP1-14. Like PD1, CTLA4 is an immune checkpoint receptor that down-modulates the amplitude of T cell activation (Pardoll 2012). Antibody blockade of CTLA4 in mice has been shown to induce antitumor immunity. The CTLA4 antibody ipilimumab has been approved for treatment of advanced melanoma and is currently under development for the treatment of various other cancer types including prostate and lung cancers, while the CTLA4 antibody tremelimumab is currently under development for the treatment of melanoma (Grosso & Jure-Kunkel 2013).

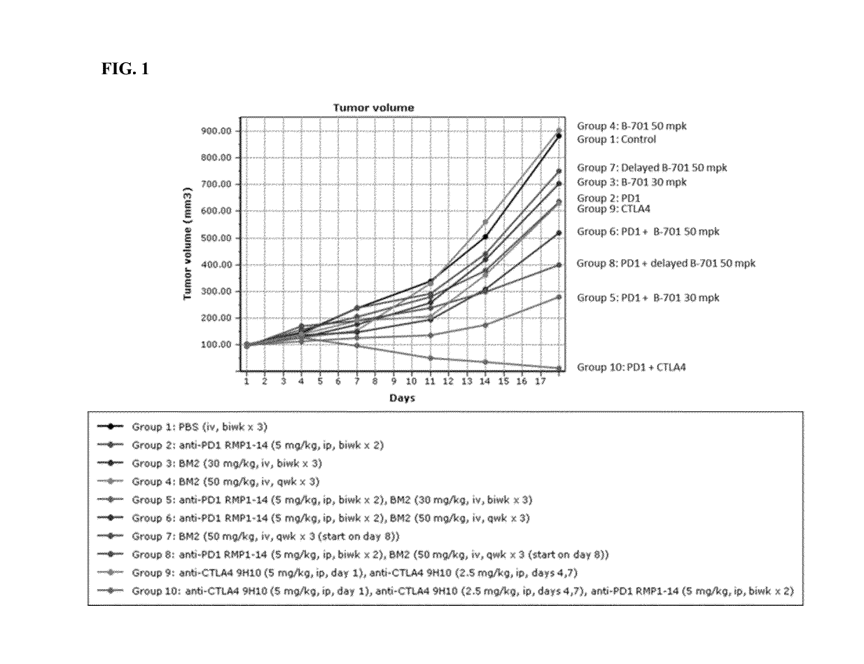

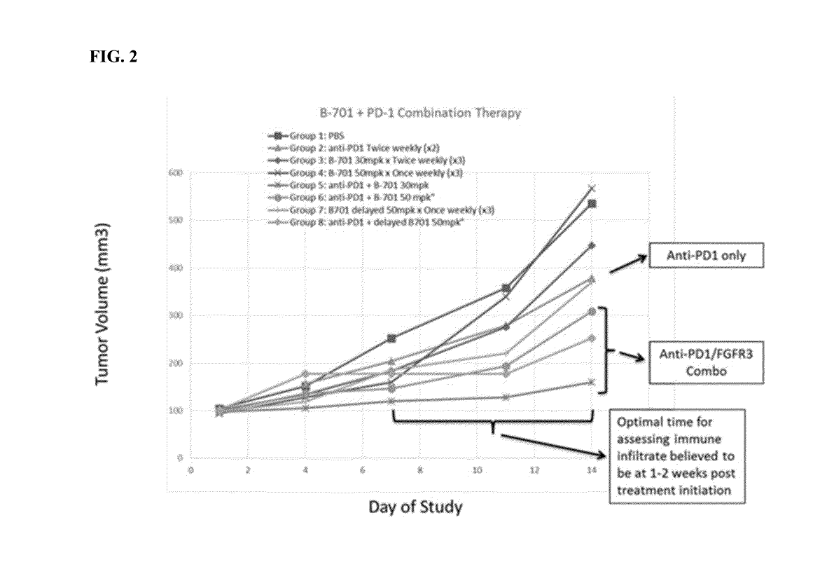

Tumor volume was monitored twice weekly using caliper measurements, and the results from all animals on study D18 are summarized in FIG. 1. Weekly administration of 50 mg/kg B-701 initiated at the start of the treatment period had little effect on tumor growth (Group 4), while delayed weekly administration of 50 mg/kg and bi-weekly administration of 30 mg/kg B-701 both slowed tumor growth to a degree similar to that observed for RMP1-14 alone (compare Groups 7 and 3 to Group 2). FIG. 2 is a snapshot of D14 study results taken after study termination, and includes data only from animals that either completed the study or were removed due to tumor progression. FIG. 2 highlights the B-701 and B-701 combination treatment groups at the 7 and 14 day dosing time points as these were selected for the subsequent study in Example 2 to examine immune cell infiltration.

FGFR3 inhibition has previously been shown to decrease immune response. Since PD1 inhibition is believed to inhibit cancer cell growth by upregulating T cell response to cancer cells, one of ordinary skill in the art would not have expected administration of an FGFR3 inhibitor to increase the anti-cancer effects of PD1. Surprisingly, however, mice administered a combination of BM2 at 30 mg/kg twice weekly or 50 mg/kg weekly or and RMP1-14 (Groups 5, 6, and 8) exhibited markedly slower tumor growth over 18 days than mice administered either antibody alone (Groups 2-4). Although more potent effects were observed when RMP1-14 was combined with 9H10, this combination is likely to be poorly tolerated in at least some patients, making alternative combinations such as FGFR3 inhibitor and PD1 inhibitor clinically important.

FGFR3 mutation and expression have been shown to be associated with a non-inflamed tumor phenotype in bladder cancer, and thus may be indicative of a tumor that is unlikely to respond to an immune checkpoint inhibitor (Sweiss 2015). Blockade of FGFR3 activity by an agent such as BM2 could improve the immune status of the tumor and make the tumor more likely to respond to checkpoint inhibitors. Thus, treatment with BM2 may be carried prior to or both prior to and concurrent with treatment with a checkpoint inhibitor.

### Example 2

### Effect of FGFR3 Antagonist on Immune Cell Infiltration

In a subsequent study, MC38 tumor cells were implanted subcutaneously into the flanks of female C57BL/6 mice that were 8 to 12 weeks of age. When tumors reached an average size of 80-120 mm³, animals were pair matched and divided into two treatment groups (n=6 mice per treatment group). Group 1 control mice received PBS via twice weekly (“biwk”) intravenous administration while Group 2 mice received B-701 twice weekly via intravenous administration at 30 mg/kg. After 7 and 14 days of treatment, three animals from each treatment group were sacrificed and tumors were collected. Half of the tumor was processed for paraffin embedding while the second half was used to prepare a single cell suspension and processed for flow cytometry the results of which are shown in Table 2.

TABLE 2
Immune infiltrate into B-701 treated tumors

| DAY 7 | | | | | DAY 14 | | | | |
|---|---|---|---|---|---|---|---|---|---|
| | | % CD8+ | % Treg | CD8/Treg | | | % CD8+ | % Treg | CD8/Treg |
| Control | Group1 | 35.6 | 6.4 | 5.6 | Control | Group1 | 12.6 | 1.5 | 8.4 |
| | Animal1 | | | | | Animal4 | | | |
| | Group1 | 56.8 | 4.1 | 13.6 | | Group1 | nd | nd | |
| | Animal2 | | | | | Animal5 | | | |
| | Group1 | 43.4 | 2.2 | 19.7 | | Group1 | 2.9 | 0.7 | 4.1 |
| | Animal3 | | | | | Animal6 | | | |
| | Ave | 44.9 | 4.2 | 13.0 | | Ave | 7.8 | 1.1 | 6.3 |
| | SD | 8.3 | 1.7 | 5.8 | | SD | 4.9 | 0.4 | 2.1 |
| B-701 | Group2 | 29.1 | 1.6 | 18.2 | B-701 | Group2 | 25.1 | 2.1 | 13.4 |
| | Animal1 | | | | | Animal4 | | | |
| | Group2 | 16.1 | 4.6 | 3.9 | | Group2 | 13.8 | 2.4 | 5.8 |
| | Animal2 | | | | | Animal5 | | | |
| | Group2 | 33.9 | 0.8 | 42.4 | | Group2 | 37.3 | 1.4 | 26.6 |
| | Animal3 | | | | | Animal6 | | | |
| | Ave | 27.0 | 2.3 | 21.5 | | Ave | 26.4 | 2.0 | 15.3 |
| | SD | 6.6 | 1.6 | 15.9 | | SD | 9.7 | 0.4 | 8.6 |

As shown in Table 2, treatment with B-701 resulted in a higher CD8+ cell to T regulatory cell ratio at both days 7 and 14 (i.e., 21.5 and 15.3, respectively), supporting the initial observation that B-701 can enhance efficacy of immune checkpoint inhibitors.

### Example 3

### Effect of FGFR3 and Immune Checkpoint Antagonists on Lung Tumor Development

FGFR3 expression was verified in the FGFR3-positive Madison 109 and Lewis Lung Carcinoma mouse lung cancer cell lines using a commercially available ELISA kit for FGFR3. Subsequently, the cell lines were expanded and 1×10⁶ Lewis Lung Carcinoma tumor cells were implanted subcutaneously into the flanks of female C57BL/6 mice that were 8 to 12 weeks of age. Additionally, 1×10⁶ Madison109 tumor cells were implanted subcutaneously into the flanks of CR female BALB/c mice that were 8 to 12 weeks of age.

When tumors reached an average size of 100-200 mm³, animals were pair matched and treatment was initiated as described in Table 3 for mice bearing Lewis Lung Carcinoma tumors and Table 4 for mice bearing Madison109 tumors. Tumors were measured using calipers twice weekly. After 7 and 14 days of treatment, three mice from each group were sacrificed and tumors were processed for histology (half of each tumor was embedded in paraffin and the other half was frozen in Optimal Cutting Temperature (O.C.T.) Compound). The remaining animals were dosed as indicated and sacrificed at day 21 (Madison 109) or day 22 (Lewis Lung Carcinoma).

TABLE 3
Lewis Lung Carcinoma treatment regimen

| | | Regimen 1 | | | | | Regimen 2 | | | | |
|---|---|---|---|---|---|---|---|---|---|---|---|
| Gr. | N | Agent | Vehicle | mg/kg | Route | Schedule | Agent | Vehicle | mg/kg | Route | Schedule |
| 1^{#} | 10 | PBS | | — | iv | biwk × 3 | — | — | — | — | — |
| 2 | 10 | BM2 | | 30 | iv | biwk × 3 | — | — | — | — | — |
| 3 | 10 | anti- | | 100* | iv | biwk × 3 | — | — | — | — | — |
| | | PD1 | | | | | | | | | |
| | | RMP1- | | | | | | | | | |
| | | 14 | | | | | | | | | |
| 4 | 10 | BM2 | | 30 | iv | biwk × 3 | anti- | | 100* | ip | biwk × 3 |
| | | | | | | | PD1 | | | | |
| | | | | | | | RMP1- | | | | |
| | | | | | | | 14 | | | | |

^{#}= Control Group
*= ug/animal

TABLE 4
Madison 109 treatment regimen

| | | Regimen 1 | | | | | Regimen 2 | | | | |
|---|---|---|---|---|---|---|---|---|---|---|---|
| Gr. | N | Agent | Vehicle | mg/kg | Route | Schedule | Agent | Vehicle | mg/kg | Route | Schedule |
| 1^{#} | 10 | PBS | | — | iv | biwk × 2 | — | — | — | — | — |
| 2 | 10 | BM2 | | 30 | iv | biwk × 2 | — | — | — | — | — |
| 3 | 10 | anti- | | 100* | ip | biwk × 2 | — | — | — | — | — |
| | | PD1 | | | | | | | | | |
| | | RMP1- | | | | | | | | | |
| | | 14 | | | | | | | | | |
| 4 | 10 | BM2 | | 30 | iv | biwk × 2 | Anti- | | 100* | ip | biwk × 2 |
| | | | | | | | PD1 | | | | |
| | | | | | | | RMP1- | | | | |
| | | | | | | | 14 | | | | |

^{#}= Control Group
*= ug/animal

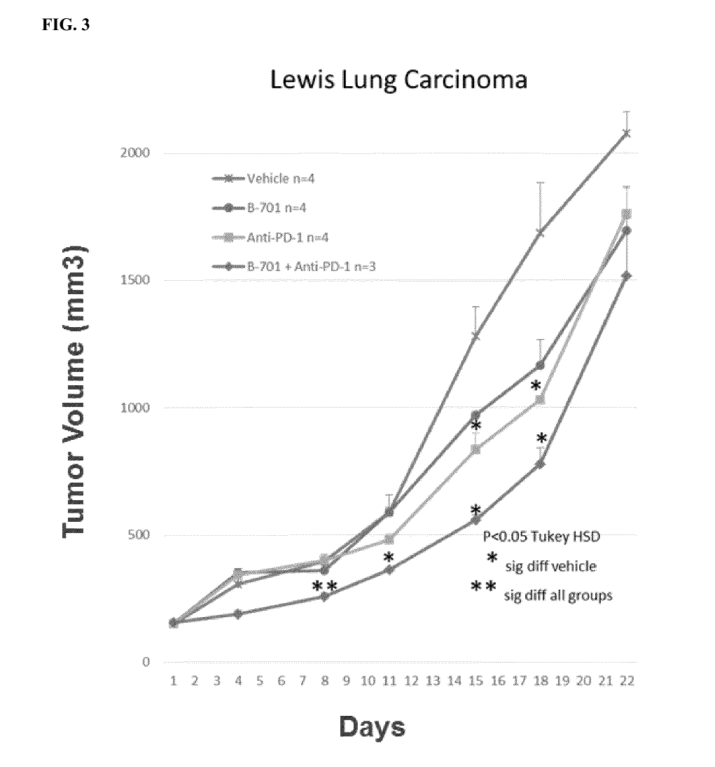

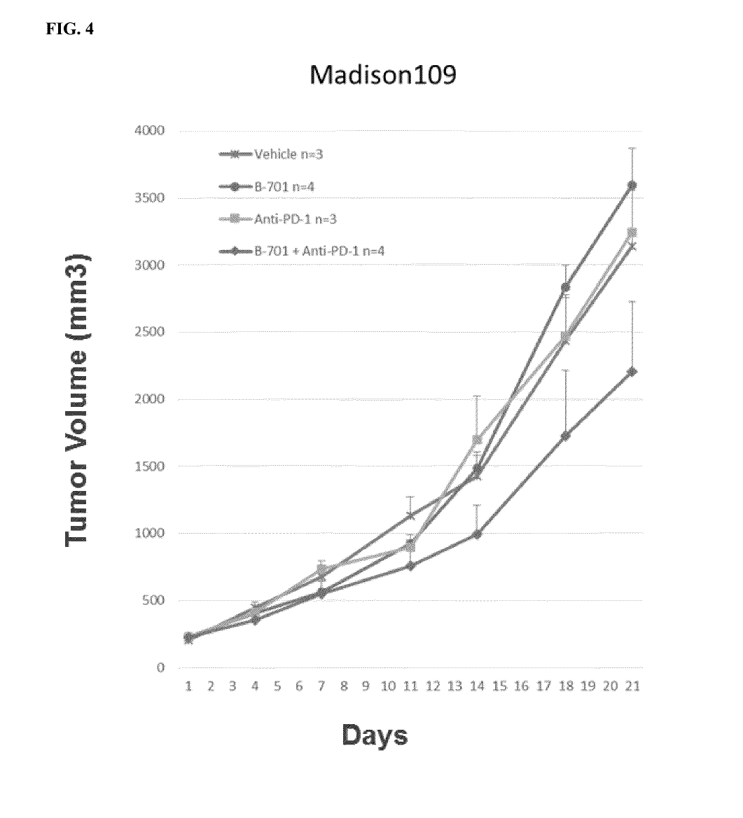

Tumor growth curves were derived from data for animals that completed the entire study (see FIG. 3 (Lewis Lung Carcinoma tumor bearing mice) and FIG. 4 (Madison 109 tumor bearing mice)). As set forth in FIGS. 3 and 4, in both studies, the greatest efficacy was observed when anti-PD-1 and B-701 were combined, further supporting that antagonism of FGFR3 enhances the effect of immune checkpoint inhibition. In the case of the Lewis Lung Carcinoma study, the combined treatment actually resulted in significantly better tumor growth inhibition at day 8 of the study (see FIG. 3, line with diamonds). Additionally, at days 8 and 11 of the Lewis Lung Carcinoma study, combination treatment was the only regimen that resulted in tumor growth suppression significant from the vehicle group (see FIG. 3, line with diamonds).

As stated above, the foregoing is merely intended to illustrate various embodiments of the present invention. The specific modifications discussed above are not to be construed as limitations on the scope of the invention. It will be apparent to one skilled in the art that various equivalents, changes, and modifications may be made without departing from the scope of the invention, and it is understood that such equivalent embodiments are to be included herein. All references cited herein are incorporated by reference as if fully set forth herein.

### REFERENCES

1. Bai et al. Cancer Res 70:7630 (2010)
2. Bumbaca et al. MAbs 3:376 (2011)
3. Brooks et al. Clin Cancer Res 18:1855-1862 (2012)
4. Grosso & Jure-Kunkel Cancer Immun 13:5 (2013)
5. Lafitte et al. Mol Cancer 12:83 (2013)
6. Pardoll Nature Reviews Cancer 12:252-264 (2012)
7. Sweiss et al. “Molecular drivers of the non-T cell-inflamed tumor microenvironment in urothelial bladder cancer” J Clin Oncol (Meeting Abstracts) 33(15) suppl (May 20 Supplement) 4511 (2015)
8. Zhao et al. Clin Cancer Res 16:5750 (2010)

What is claimed is:

1. A method of treating a solid tumor in a subject in need thereof comprising administering a therapeutically effective amount of an FGFR3 inhibitor in combination with a therapeutically effective amount of a PD1 inhibitor.

2. The method of claim 1, wherein the FGFR3 inhibitor is an antagonistic FGFR3 antibody.

3. The method of claim 2, wherein the antagonistic FGFR3 antibody comprises CDR-H1 comprising the amino acid sequence set forth in SEQ ID NO:1, CDR-H2 comprising the amino acid sequence set forth in SEQ ID NO:2, and CDR-H3 comprising the amino acid sequence set forth in SEQ ID NO:3.

4. The method of claim 3, wherein the antagonistic FGFR3 antibody comprises a heavy chain variable region comprising the amino acid sequence set forth in SEQ ID NO:7.

5. The method of claim 2, wherein the antagonistic FGFR3 antibody comprises CDR-L1 comprising the amino acid sequence set forth in SEQ ID NO:4, CDR-L2 comprising the amino acid sequence set forth in SEQ ID NO:5, and CDR-L3 comprising the amino acid sequence set forth in SEQ ID NO:6.

6. The method of claim 5, wherein the antagonistic FGFR3 antibody comprises a light chain variable region comprising the amino acid sequence set forth in SEQ ID NO:8.

7. The method of claim 1, wherein the PD1 inhibitor is an antagonistic PD1 antibody.

8. The method of claim 7, wherein the antagonistic PD1 antibody is selected from the group consisting of nivolumab, pembrolizumab, CT-011, MEDI-0680, and RMP1-14.

9. The method of claim 1, wherein the PD1 inhibitor is an antagonistic PD1 ligand antibody.

10. The method of claim 9, wherein the antagonistic PD1 ligand antibody is selected from the group consisting of MEDI-4736, RG7446, BMS-936559, MSB0010718C, and MPDL3280A.

11. A pharmaceutical composition comprising an FGFR3 inhibitor and a PD1 inhibitor.

12. The composition of claim 11, further comprising a pharmaceutically acceptable carrier.

13. The composition of claim 11, wherein the FGFR3 inhibitor is an antagonistic FGFR3 antibody.

14. The composition of claim 13, wherein the antagonistic FGFR3 antibody comprises CDR-H1 comprising the amino acid sequence set forth in SEQ ID NO:1, CDR-H2 comprising the amino acid sequence set forth in SEQ ID NO:2, and CDR-H3 comprising the amino acid sequence set forth in SEQ ID NO:3.

15. The composition of claim 14, wherein the antagonistic FGFR3 antibody comprises a heavy chain variable region comprising the amino acid sequence set forth in SEQ ID NO:7.

16. The composition of claim 13, wherein the antagonistic FGFR3 antibody comprises CDR-L1 comprising the amino acid sequence set forth in SEQ ID NO:4, CDR-L2 comprising the amino acid sequence set forth in SEQ ID NO:5, and CDR-L3 comprising the amino acid sequence set forth in SEQ ID NO:6.

17. The composition of claim 16, wherein the antagonistic FGFR3 antibody comprises a light chain variable region comprising the amino acid sequence set forth in SEQ ID NO:8.

18. The composition of claim 11, wherein the PD1 inhibitor is an antagonistic PD1 antibody.

19. The composition of claim 18, wherein the antagonistic PD1 antibody is selected from the group consisting of nivolumab, pembrolizumab, CT-011, MEDI-0680, and RMP1-14.

20. The composition of claim 11, wherein the PD1 inhibitor is an antagonistic PD1 ligand antibody.

21. The composition of claim 20, wherein the antagonistic PD1 ligand antibody is selected from the group consisting of MEDI-4736, RG7446, BMS-936559, MSB0010718C, and MPDL3280A.
